(19) United States
(12) Patent Application Publication
(10) Pub. No.: US 2009/0326612 A1
(43) Pub. Date: Dec. 31, 2009

(54) Electronic biofeedback stimulation device

(75) Inventor: Michael J. Distler, Kemah, TX (US)

Correspondence Address: MICHAEL J. DISTLER, 2209 MONTERREY, KEMAH, TX, 77565, US

(73) Assignee: Michael J. Distler, Kemah, TX (US)

(21) Appl. No.: 12/215,341

(22) Filed: Jun. 26, 2008

(51) Int. Cl. A61N 1/36

(52) U.S. Cl. 607/62

(57) ABSTRACT

A Biofeedback treatment device providing electrical stimulation to a patients skin. A microprocessor generates a control signal out to the patients skin. The return signal from the patients feeds into the microprocessor. Circuitry within the biofeedback stimulation device analyzes the condition of said patients inflammation.

During the treatment there are two indicators of condition.
1. A visual indication via display screen.
2. An audible indication via internal piezo of the biofeedback treatment progress of said patient.

### FIELD OF INVENTION

The invention relates to the field of pain management systems, and particularly to Biofeedback Stimulation Devices.

### BACKGROUND OF THE INVENTION

There are many people with injuries and ailments, treatable with this invention. Examples include backpain, sprains, carpal tunnel syndrome, arthritis and numbness of extremities as with neuropathy, stroke, and neurological conditions such as “ADD” and macular degeneration. These are all ailments from which the human body must work to recover. They are not viruses, infections or chemically related ailments. They are not instances where surgery has proven effective such as the reattaching of bones, ligaments, or other body parts or clearing of arteries.

Energetic medicine addresses energy related ailments. There has been much research into energetic medicine, and the way the body's electric and nervous system works, dating back to the 1900s. Devices have been developed such as the rife machine, and infrared light therapies used in energetic medicine.

There are diagnostic tools, such as the Mead machines, which measure resistance in the body's energetic pathways called energy meridians.

There also are treatment machines in the category of TENS* and “Electronic Acupuncture”.

### SUMMARY OF THE INVENTION

The present invention, as disclosed and described herein on one aspect thereof, comprises a Biofeedback stimulation device. A biofeedback stimulation device sends a preset waveform to the patient via electrodes of said device. The said device then receives a return signal from said patient which indicates the status of the inflamed area sent to the microprocessor. The microprocessor controls the level of therapy based on the return signals from said patient. The treatment level controlled by a signal from the microprocessor. The more inflamed area is sent less “damping” and the less inflamed area is sent more damping impulse.

### DETAILED DESCRIPTION OF THE INVENTION

The device is based on an ARM7 flash micro-controller (100). The software is written in C++ code The device uses a color graphical O.L.E.D. display (101) This organic O.L.E.D. requires no backlight and has a wide viewing angle. On/off control and other functions are set by a custom membrane type keypad with joystick type arrangement. The keypad has two L.E.D.S installed to indicate power output level and battery charge status (102).

The device is powered by a 3.7 volt lithium ion battery pack (103). There is a charge control circuit (104) for controlling the charge rate of the battery pack and also for detecting the desk top drop in charger (105) or the usb charger. The device has a low voltage dropout circuit (106) to turn the unit off if the battery voltage goes too low. This maintains accuracy of the unit based on voltage level There is a bar graph indicator for the battery condition. This is a separate microcontroller (107) and it recalibrates for each new charge and also. If a battery is changed it will recalibrate for the replacement battery. The battery is internally fused and the p.c.b. is also fused.

The ARM7 flash microcontroller has 512 k of onboard R.A.M. allowing for storage of patient treatment data. There is an additional 8 mb of dataflash on the p.c.b. to add more memory capacity (108).

The Delta Scanner devices have different modes of operation and can be flashed with different software depending on the intent of the user. All models have an analysis mode and treatment mode. These modes have different bipolar pulses which vary from 5 uS to 500 Us and are generated by the micro-controller (100) and sent to the pulse width modulation circuit (112) and men to the pulse driver circuit (113). These pulses are then sent to the electrodes (114) or the external electrode jack (115) for use of various probes. “Analyze” mode frequency is 593 Hz “Treatment” modes very from 32 Hz to 250 Hz depending on one selected treatment. Some treatment modes have a sweeping frequency mat changes over a preset range. Amplitude of the bipolar pulse is 100 v at 100% power level setting. The unit has a damping circuit to create half-cycles in the bipolar output pulse. Patient data can be downloaded using the usb port (109) to a personal computer containing the Delta Scanner software Patient Management Program. Patient shock hazard protection is provided to meet safety requirements using a usb switch (110) to isolate tills port electrically when in “treatment” or “analyze” mode of operation.

There is a real time clock (111) to provide accurate date and time for patient data. This is kept active using a memory capacitor.

The Delta Scanner has a piezo (116) that provides a unique Audible output that can be used to indicate treatment progress.

All references are show to FIG. 1 block diagram.

### BRIEF DESCRIPTION OF THE DRAWINGS

FIG. 1 DELTA SCANNER block diagram flowchart of the present invention.

FIG. 2,3 and 4 schematic diagram of the DELTA SCANNER.

FIG. 5 software flowchart for the microprocessor.

FIG. 6 software flowchart for analyze mode feedback section of the DELTA SCANNER.

FIG. 7 Skin conductance graph of DELTA SCANNER.

FIG. 8 waveform tracings single pulse showing analyze mode for DELTA SCANNER.

FIG. 9a,9b,9c waveform tracings showing typical treatment

mode outputs.

FIG. 10 Delta Scanner device showing view of unit inserted in a desk top charger with external probe.

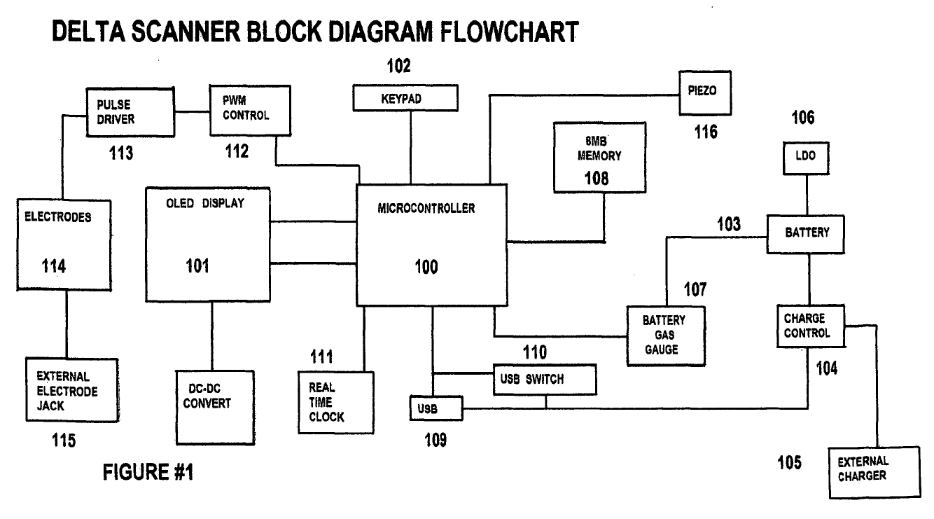

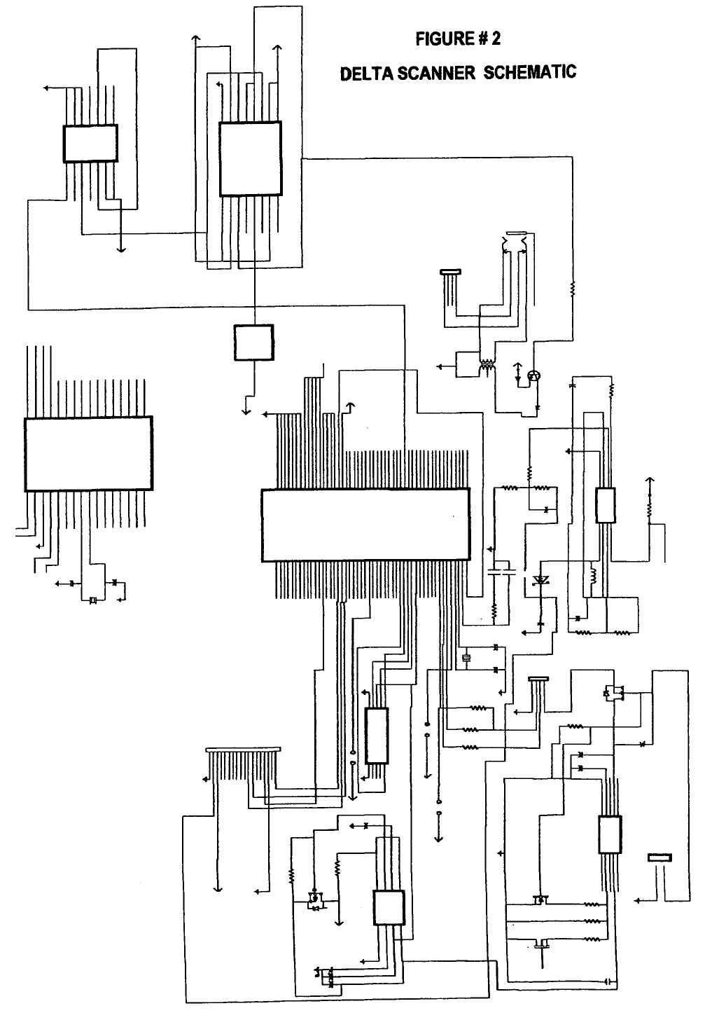

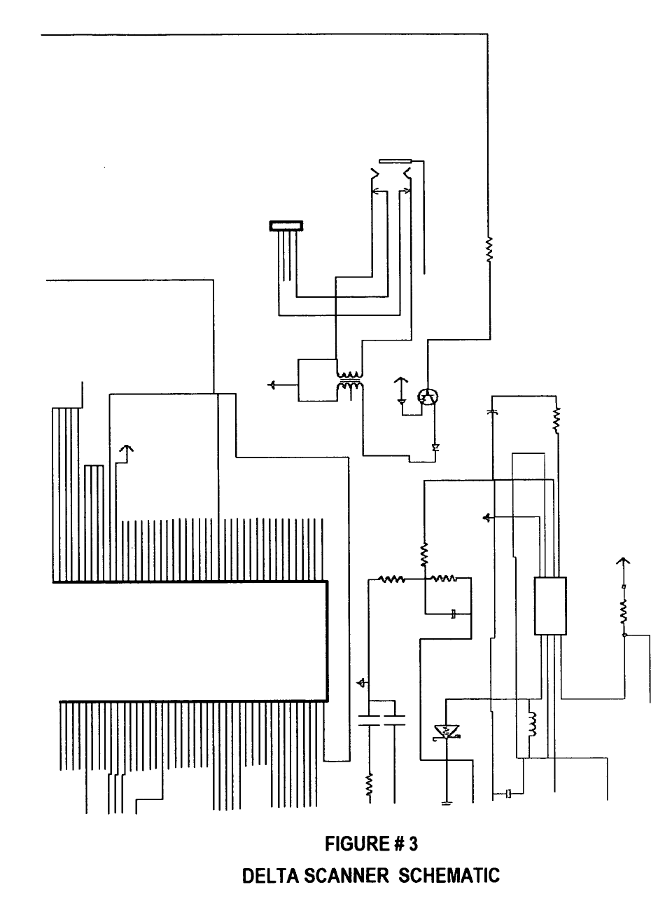

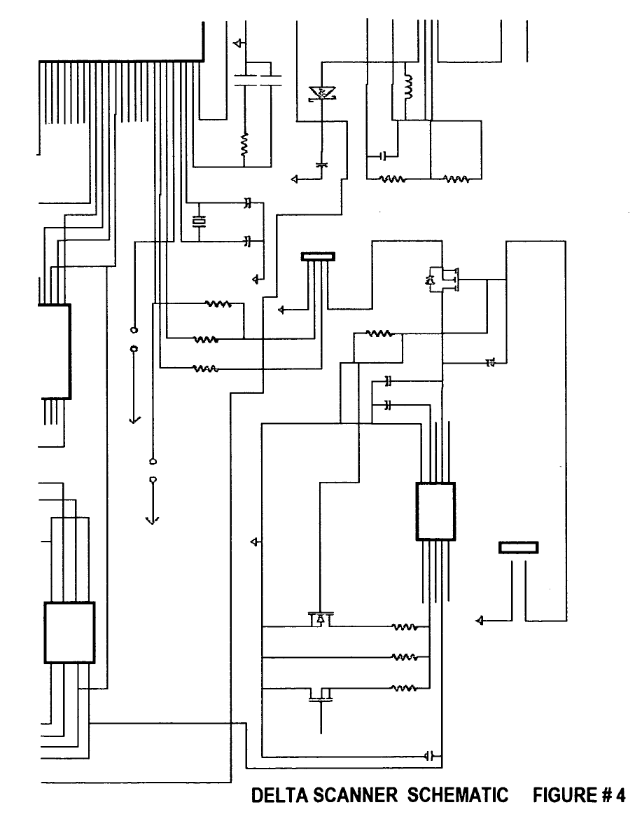

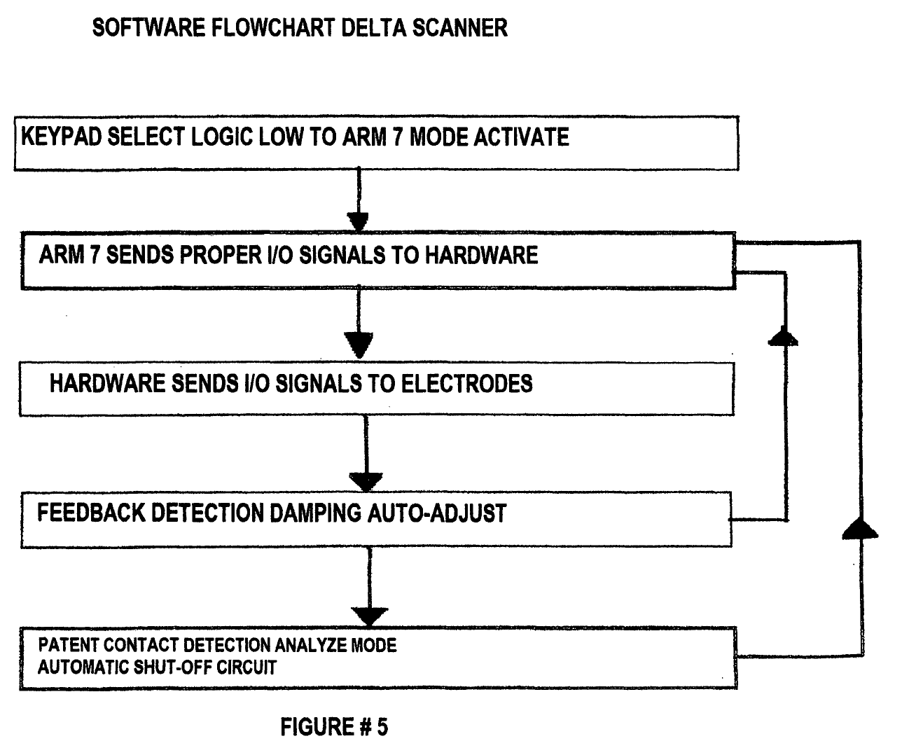

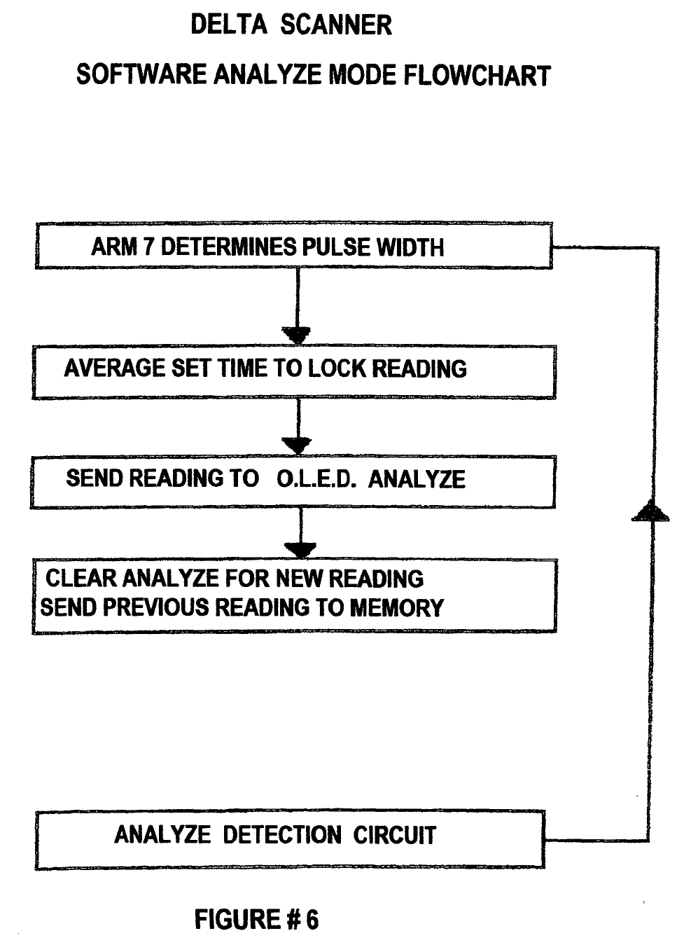

1. A Biofeedback stimulation device, comprised of an external housing adapted to be grasped generally at a base portion thereof by a users hand, a plurality of electrodes placed on a distal of said housing, an internal pulse generating circuitry adapted to generate a stream of electrical impulses between said plurality of electrodes, and a distal surface adapted for contact with a patients skin thereby electrically stimulating said patients neural tissue in a localized are.

2. A Biofeedback stimulation device of claim 1, wherein the circuitry further enables a modulated application of electrical impulse signal to the body of the individual.

3. The biofeedback stimulation of claim 1, wherein the circuitry further comprises; first circuitry for generating and detecting the electrical stimulation signal and second, circuitry for reacting to patients skin conductance.

4. The biofeedback stimulation device relies on comparing the signal out and the signal return modulated by the patients return pulses within his system.

5. The biofeedback stimulation device monitors the output signal and its return. It is controlled by a flash microprocessor for dampening. This relies on the degree of the individually generated feedback response signal whenever the electrical stimulation signal is applied.

6. The Biofeedback stimulation device is capable of multi-function due to its microprocessor. There are numerous internal pulses which are controllable by the processor, adapting to the conductance of said patients skin.

7. The Biofeedback stimulation device has a graphical color O.L.E.D. (organic color light emitting diode). It requires no backlighting. Universal language fonts available on the microprocessor flash for different applications of the device.

8. The Biofeedback device contains a lithium ion rechargeable battery. Charging can be done in two ways. #1, “drop-in” recharge station and #2, via the usb port on said device. When a usb cable is attached to said device, the usb port is electrically isolated, not allowing any electrical leakage or ground fault from the device when said device is in the “analyze” or “treatment” modes.

9. Upload and download capability via the usb port for compute documentation of said patient's treatment history.

10. Secure usb upload and download capability of patients treatment history.

11. Patient's treatment history is stored within the device via flash memory, it can he downloaded to a computer. It can also be stored on an external flash drive.

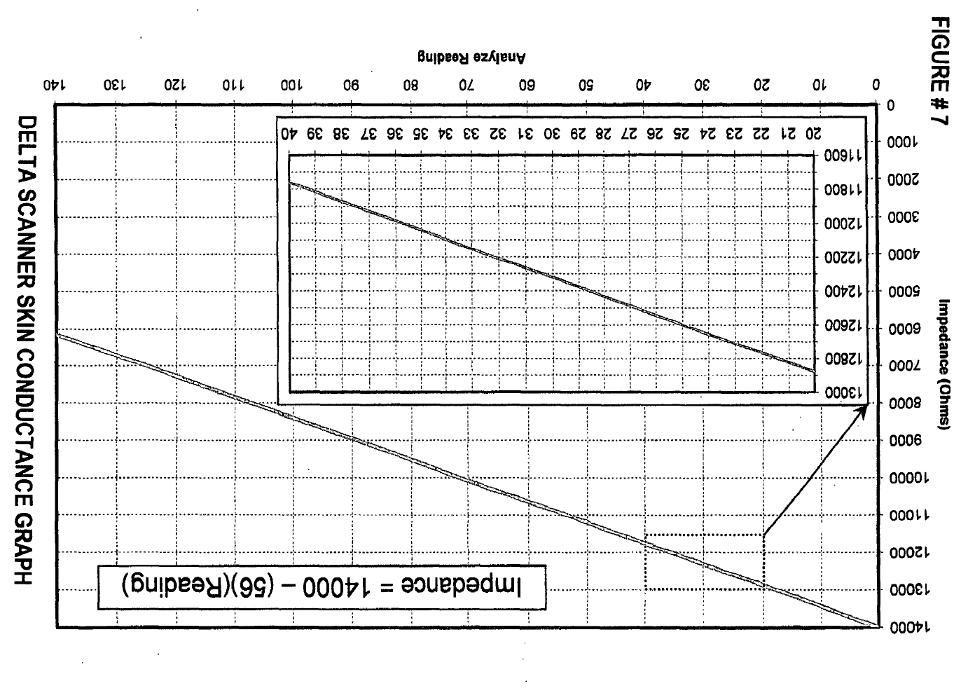

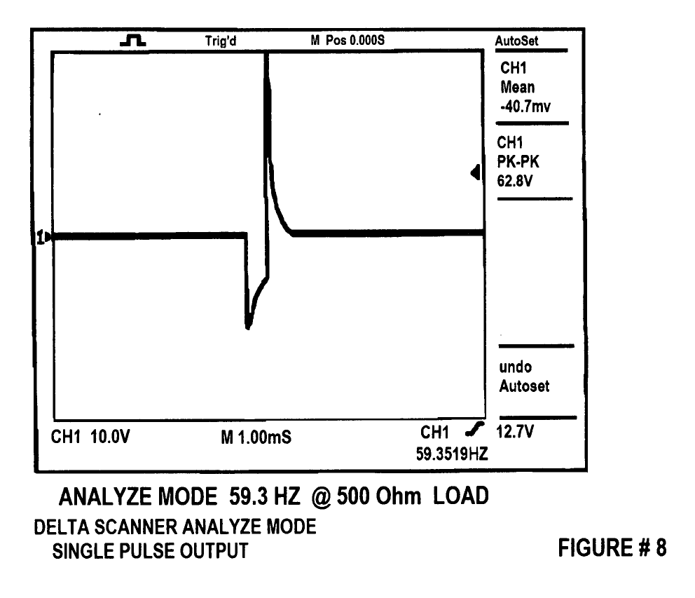

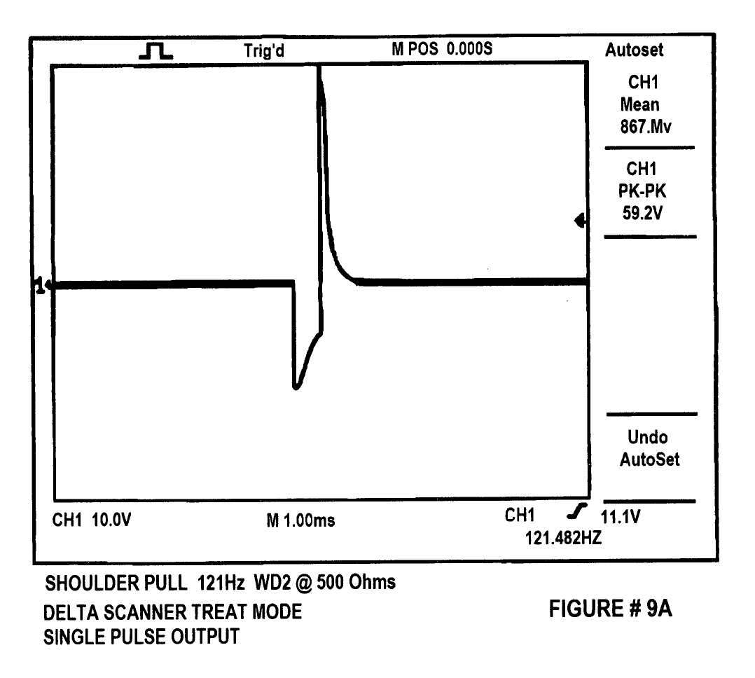

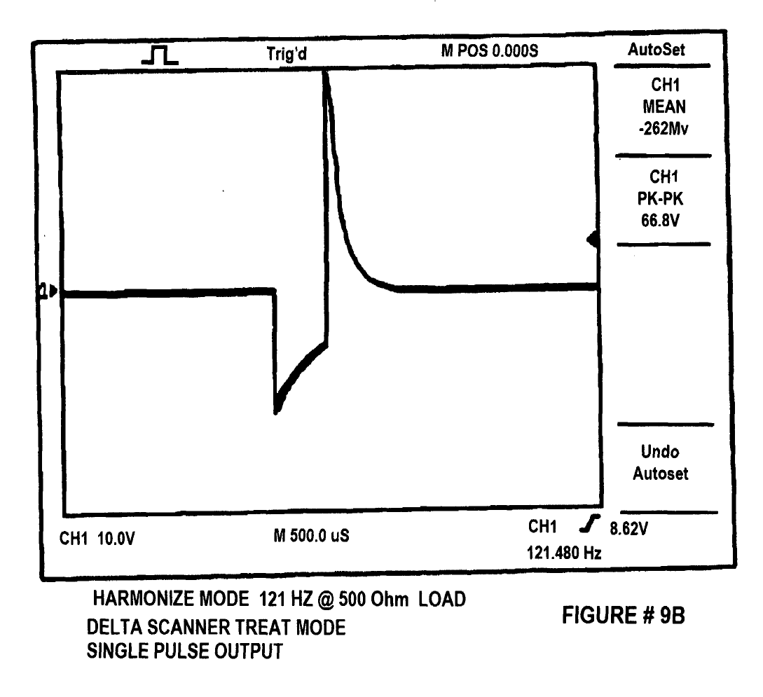

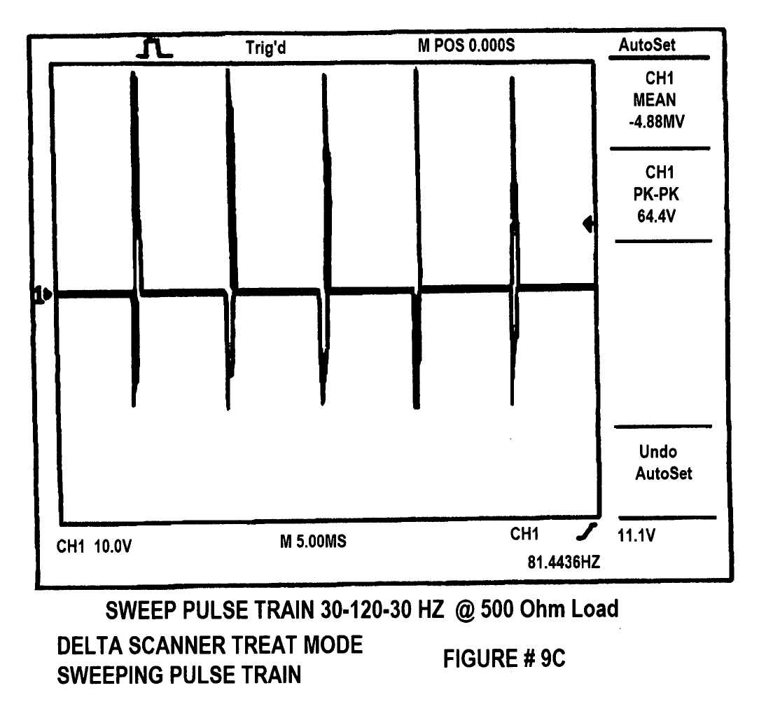

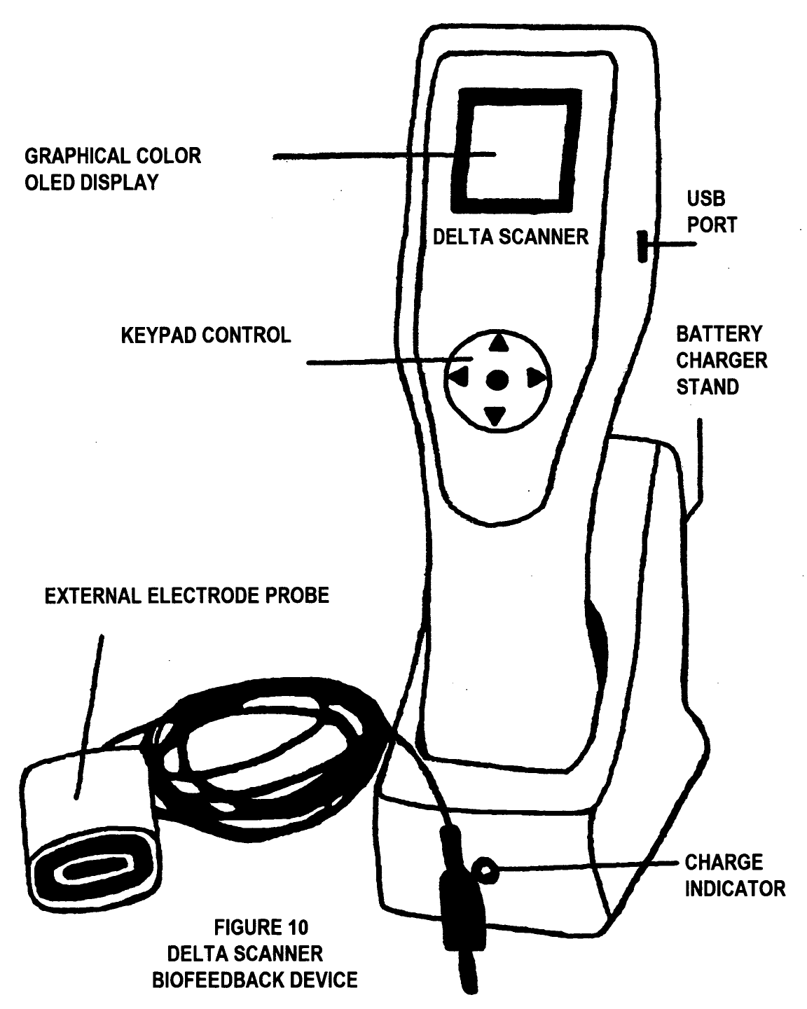

12. FIG. 7 of the Biofeedback stimulation device represents the patient's skin conductance as indicated in the analyze mode when electrodes are in contact with the patients skin.

13. The Biofeedback stimulation device emits an audio feedback sound to the operator via a piezo as an indicator of the degree of electrical stimulation of skin conductance of the localized area at the electrodes applied to the patient's skin. The audible sound is heard during ail three modes of operation: analyze, treat and feedback.

14. The device transmits an electro-positive signal. The inflamed skin is “electro-negative”. Application of the device produces a magnetic attraction between the skin and the electrodes which causes a “dragging” effect on the skin indicating to the operator the degree of inflammation of area of contact. This also is visually indicated on the O.L.E.D. display. The emitted audio sound changes its frequency during treatment. A highly inflamed area will produce a low frequency sound which also indicates the increased magnetic attraction of skin to the electrodes causing the “dragging” effect of the device. A less inflamed area will produce a higher frequency sound to the operator and a less “dragging” effect.
